(19) United States
(12) Patent Application Publication
(10) Pub. No.: US 2021/0262041 A1
(43) Pub. Date: Aug. 26, 2021

(54) COMBINED EXPRESSION PATTERN OF SATB FAMILY CHROMATIN ORGANIZERS AS IMPROVED BIOMARKER TOOL FOR CANCER PROGNOSIS

(71) Applicant: INDIAN INSTITUTE OF SCIENCE EDUCATION AND RESEARCH, Pune, Maharashtra (IN)

(72) Inventors: Sanjeev GALANDE, Pune (IN); Rutika NAIK, Pune (IN)

(21) Appl. No.: 17/266,206

(22) PCT Filed: Aug. 8, 2019
(86) PCT No.: PCT/IN2019/050580

§ 371 Date: Feb. 5, 2021

(30) Foreign Application Priority Data
Aug. 8, 2018 (IN) 201821029791

(51) Int. Cl. C12Q 1/6886

(52) U.S. Cl. CPC C12Q 1/6886; C12Q 2600/112; C12Q 2600/158

(57) ABSTRACT

The present invention provides an in-vitro process for profiling a combined expression pattern of SATB (special AT-rich sequence-binding protein-1) family chromatin organizers i.e. SATB-1 and SATB-2 as a biomarker tool for cancer prognosis to assess cancer aggressiveness and it progression in an array of cancer types. The combined expression pattern of SATB is established by the method of Kaplan Meier survival analysis of a dataset of patients from a plurality of cancer types. Further, the present invention provides a prognostic kit to evaluate the progression of cancer by using the combined expression pattern of SATB-1 and SATB-2 as a biomarker tool for cancer prognosis.

### TECHNICAL FIELD OF THE INVENTION

The present invention relates to a novel process for profiling a combined expression pattern of SATB (special AT-rich sequence-binding protein-1) family chromatin organizers i.e. SATB-1 and SATB-2 as a biomarker tool for cancer prognosis.

Further, the present invention relates to a dual expression pattern of SATB proteins as an improved tool for assessment of cancer aggressiveness and as a combination of novel prognostic markers for an array of cancer types.

### BACKGROUND AND PRIOR ART OF THE INVENTION

Cancer is a leading cause of death in India and worldwide. It is a complex disease and a multistep process occurring via genetic alterations or aberrations leading to altered gene expression. There is lack of precise prognostic biomarkers to evaluate aggressiveness of the disease or cancer progression for multiple cancer types.

Cancers are diverse with respect to cell of origin, exhibit dynamic molecular expression pattern influenced by tissue microenvironment. This is a major limitation leading to failure of cancer therapy. There is a need of precise biomarkers to evaluate aggressiveness of cancer. These prognostic markers would evaluate disease prognosis and also help towards decision of cancer therapy. In this context, the role of SATB family chromatin organizers has been implicated in cancer progression.

SATB (Special AT-rich binding protein) family proteins (SATB1 and SATB2) have emerged as key regulators that integrate higher-order chromatin organization with regulation of gene expression. Studies over the past decade have elucidated specific roles of SATB1 and SATB2, two closely related members of this family individually in cancer progression. SATB family chromatin organizers play diverse and important roles in regulating the dynamic equilibrium of apoptosis, cell invasion, metastasis, proliferation, angiogenesis, and immune modulation.

Number of studies in the past decade has demonstrated strong association between expression of SATB1 with tumor aggressiveness in an array of cancer types. Mir et al. Oncogene 2016 published by the present inventors, Galande et al. have elucidated the role of SATB1 in colorectal cancer tumorigenesis and tumor progression. The authors have explained the mechanistic understanding of the regulation of SATB1 and have observed that it is necessary and sufficient to regulate the expression of β-catenin, TCF family members and multiple downstream effectors and mediators for regulation of the Wnt signaling pathway.

A reference may be made to a study by Kohwi-Shigematsu et al. titled “Cancer-associated mar binding protein” suggested SATB1 as a diagnostic marker for T cell leukemia (U.S. Pat. No. 5,624,799). Aberrant expression of SATB1 is speculated to promote lymphoma (Agrelo et al. Dev. Cell. 2009) and cutaneous T cell lymphoma (Fredholm et al. J Invest Dermatol. 2018; Sun et al. J Invest Dermatol. 2018).

Further, Kohwi-Shigematsu et al. in US Patent Publication No. 2015/0323536 has implicated the role of SATB1 in tumour metastasis in the study titled “SATB1: A Determinant of Morphogenesis and Tumor Metastasis”.

Kohwi-Shigematsu et al. in US Patent Publication No.2017/067125 also described use of long non-coding RNA targeting SATB1 as a therapeutic target for breast cancer cells, this study is titled ‘Long Non-Coding RNA Expressed in Aggressive Cancer’. Zheng et al showed that aberrant expression of SATB1 leads to breast tumour progression and metastasis.

A reference may also be made to US 2018/0010195, wherein Srivastava. S has described the use of SATB1 as one of the marker in the panel for evaluation of cancer prognosis and diagnosis. This study is titled as “Compositions and methods for monitoring, diagnosis, prognosis, detection and treatment of cancer”.

Additionally, a number of studies have correlated expression of SATB1 with aggressiveness of cancer in case of nasopharyngeal (Deng et al., Int. J. Clin. Exp. Pathol. 2014), bladder (Han et al., Tumour Biol. 2013), prostate (Mao et al. J. Transl. Med. 2013; Wang et al. Exp Ther Med. 2018), lung (Selinger et al., J. Thorac. Oncol. 2011), ovarian (Xiang et al., Oncol. Lett. 2012), liver (Tu et al., Liver Int. 2012) and glioma (Chu et al., J. Transl. Med. 2012).

Elebro et al., (J. Transl. Med. 2014) have proposed SATB1 as a prognostic and predictive biomarker for both intestinal type and pancreato-biliary type periampullary adenocarcinomas, including pancreatic cancer.

Feng et al. (Exp Ther Med. 2018) and Cheng et al. (PLoS One, 2014) have showed a strong association of SATB1 with endometrial carcinoma and renal cancer respectively.

With respect to SATB2, its expression is correlated with aggressiveness of colorectal cancer by Wang et al. (J. Pathol. 2009), bone cancer by Liu et al. (PLoS One, 2012), head and neck cancer by Seong et al. (Oncogene, 2015) demonstrating its cancer specific role.

A study by Uhlén et al. titled “Use of Protein SATB2 as a marker for Colorectal Cancer” proposed SATB2 alone as a diagnostic marker for colorectal cancer (US patent-8465934).

All of the studies mentioned above have considered SATB1 or SATB2 individually and correlated its expression with tumour aggressiveness. However, these studies have provided no inclination whatsoever to address the importance of the dual expression pattern of SATB family chromatin organizers towards cancer progression and its role in the prognosis of an array of cancer types.

### OBJECT OF THE INVENTION

An object of the present invention is to evaluate the dual expression of SATB family chromatin organizers in an array of cancer types and validate its correlation with patient survival using TCGA (The Cancer Genome Atlas) cancer patient survival data to establish cancer progression.

Another object of the present invention is to define a balance between the expression patterns of SATB family chromatin organizers, which is tumor type specific and serves as a determinant of survival of the cancer patient.

### SUMMARY OF THE INVENTION

SATB family proteins have emerged as key regulators that integrate higher-order chromatin organization with regulation of gene expression. Studies over past decade have elucidated specific roles of SATB1 and SATB2, two closely related members of this family, in cancer progression. However, all the studies performed till date have considered SATB1 and SATB2 in isolation.

In an aspect, the present invention provides an in-vitro method for evaluating the progression and aggressiveness of cancer in a subject characterized by evaluating the combined expression patterns of SATB (Special AT-rich Sequence-Binding Protein) chromatin organizers, i.e., SATB-1 and SATB-2, which comprises;
i. culturing tumorigenic cell cultures isolated from a primary tumor tissue, and
ii. evaluating the combined expression levels of SATB-1 and SATB-2 chromatin organizers in the said cultures by RNA isolation and sequencing and quantitative RT-PCR.

In another aspect, the present invention provides a process for evaluating the dual expression patterns of SATB family chromatin organizers towards cancer prognosis comprising;
(a) performing Kaplan Meier survival analysis of TCGA (The Cancer Genome Atlas) dataset of patients from a plurality of cancer types, and
(b) comparing patient survival data to gene expression obtained through RNA sequencing of tumor samples of plurality of cancer types from TCGA to evaluate the significance of SATB1 and SATB2 chromatin organizers towards cancer prognosis.

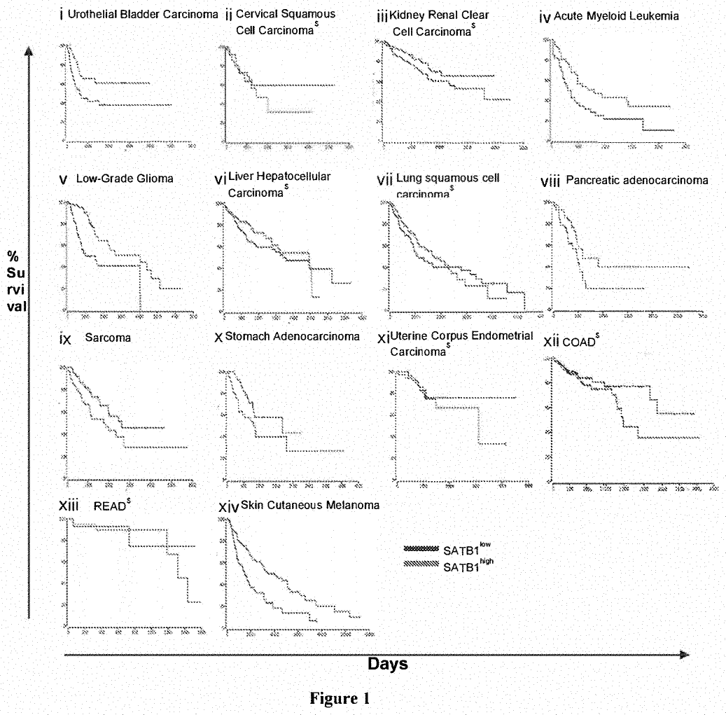

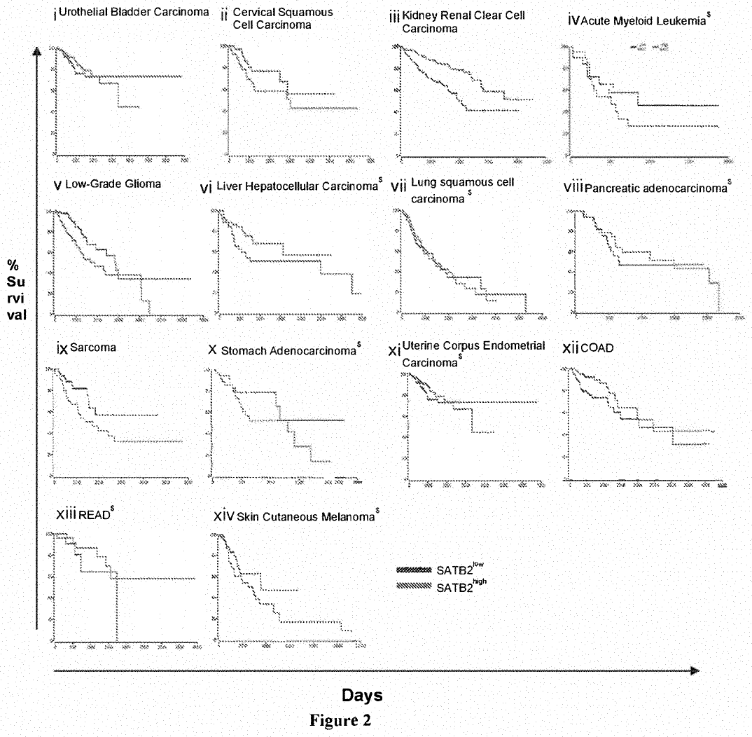

In yet another aspect, the present invention provides a prognosis kit for evaluating the progression of cancer in a subject by estimating the dual expression pattern of SATB family chromatin organizers towards cancer prognosis, the said kit comprising a chart indicating the expression levels of SATB-1 and SATB-2 chromatin organizers in a plurality of cancer cell types, wherein the said chart comprises expression levels of SATB-1 and SATB-2 chromatin organizers in a plurality of cancer cell types for comparison of combined expression levels of SATB-1 and SATB-2 proteins in patient tumor samples;
(i) combined increased expression of SATB1 and SATB2 is strongly correlated with poor patient survival for cervical squamous cell carcinoma, sarcoma and stomach adenocarcinoma;
(ii) SATB1 expression correlated negatively with SATB2 which culminates in poor patient survival for colon adenocarcinoma (COAD), urothelial bladder carcinoma rectal adenocarcinoma (READ), lung squamous cell carcinoma, uterine corpus endometrial carcinoma and liver hepatocellular carcinoma; and
(iii) increased expression of SATB2 and decreased expression of SATB1 is determinant of poor patient survival for acute myeloid leukaemia, low-grade glioma, skin cutaneous melanoma and pancreatic adenocarcinoma (FIG. 1; FIG. 2).

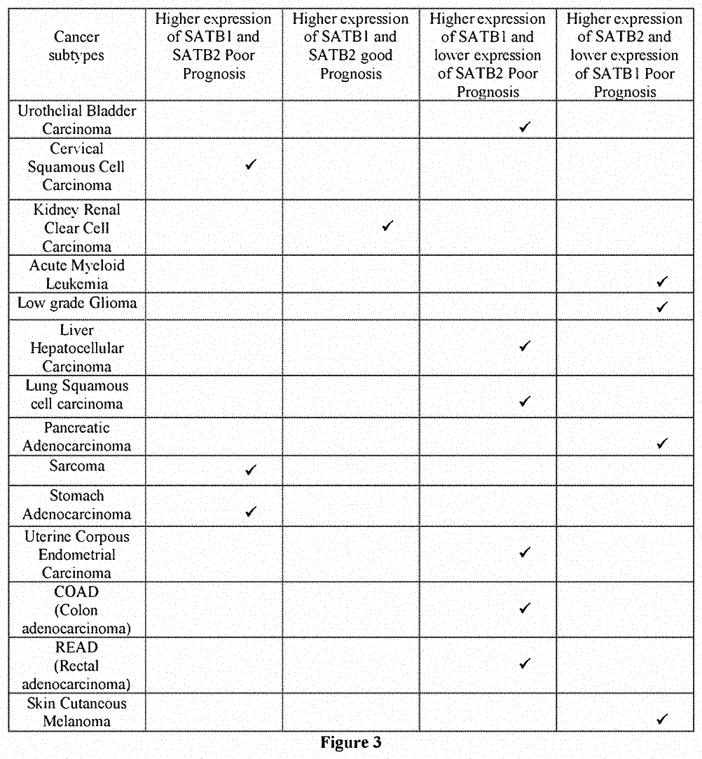

The said chart indicating the combined expression levels of SATB1 and SATB2 in a plurality of cancer cell types are defined in FIG. 3.

In another aspect, the present invention provides tissue specific expression pattern of the SATB1 and SATB2 chromatin organizers in 3D spheroid cultures and 2D cell lines, wherein the combined expression of the said SATB chromatin family organizers serve as a highly reliable tool to precisely predict tumor aggressiveness.

The present invention also validates functional significance of expression of SATB family chromatin organizers. Accordingly, 3-Dimensional (3D) spheroid cultures which are an in vitro model for tumor regenerative cells were established.

The expression pattern of SATB family chromatin organizers more preferably in colorectal cancer cell line generated spheroids (3D culture) and cell lines (2D culture) was profiled. An upregulation of SATB1 in spheroids and its down-regulation in 2D cultures was observed, and SATB2 was highly expressed in 2D cultures compared to 3D spheroid cultures, therefore, suggesting the role of dynamic expression of SATB family chromatin organizers in regulating cellular plasticity.

Therefore, the present invention convincingly demonstrates that there exists a dynamic balance between expression patterns of SATB1 and SATB2 chromatin organizers, which is tumor type specific and serves as an accurate determinant of cancer patient survival.

### DETAILED DESCRIPTION OF DRAWINGS

FIG. 1: Kaplan Meier analysis plots for expression of SATB1 in various cancer types. The red line indicates higher expression of SATB1 whereas blue line indicates lower expression of SATB1. Data are plotted for Urothelial bladder carcinoma; cervical squamous cell carcinoma; glioblastoma; kidney renal clear cell carcinoma; kidney renal papillary cell carcinoma; acute myeloid leukemia; low-grade glioma; liver hepatocellular carcinoma; lung adenocarcinoma; lung squamous cell carcinoma; ovarian Cancer; pancreatic adenocarcinoma; sarcoma; skin cutaneous melanoma; stomach adenocarcinoma; uterine corpus endometrial carcinoma; colon adenocarcinoma; rectal adenocarcinoma; breast invasive carcinoma. Statistical significance was calculated using logrank p-value≤00.5; $ indicates dataset with lower patient numbers with p value≤0.1.

FIG. 2: Kaplan Meier analysis plots for expression of SATB2 in various cancer types. The red line indicates higher expression of SATB2 whereas blue line indicates lower expression of SATB2. Data are plotted for Urothelial Bladder Carcinoma; Cervical Squamous Cell Carcinoma; Glioblastoma; Kidney Renal Clear Cell Carcinoma; Kidney renal papillary cell carcinoma; Kidney renal papillary cell carcinoma; Acute Myeloid Leukemia; Low-Grade Glioma; Liver Hepatocellular Carcinoma; Lung Adenocarcinoma; Lung squamous cell carcinoma; Ovarian Cancer; Pancreatic adenocarcinoma; Sarcoma; skin cutaneous melanoma; Stomach Adenocarcinoma; Uterine Corpus Endometrial Carcinoma; Colon Adenocarcinoma; Rectal Adenocarcinoma; Breast Invasive Carcinoma. Statistical significance was calculated using logrank p-value≤00.5; $ indicates dataset with lower patient numbers with p value≤0.1.

FIG. 3 depicts Correlation of expression of SATB family chromatin organizers with patient survival across cancer types.

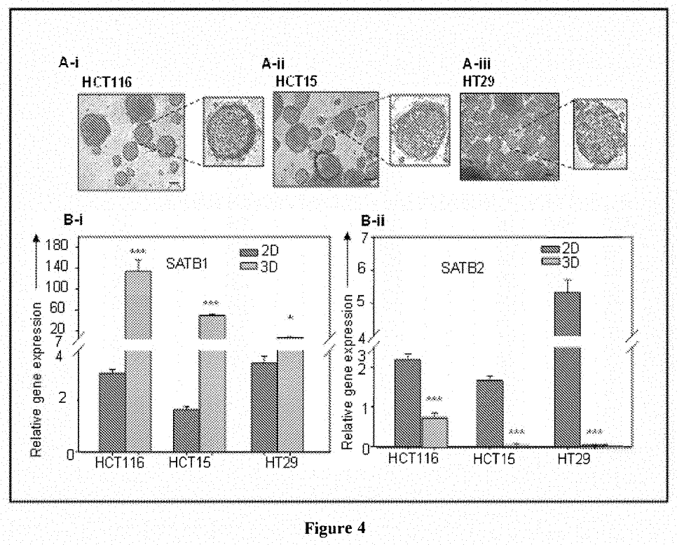

FIG. 4: SATB family chromatin organizers exhibit dynamic regulation in 2D vs 3D (spheroid) cultures. A. Representative image of 7^{th} day 3D (Spheroids) generated from colorectal cancer cell line A-i HCT116, A-ii HT29, A-iii HCT15, B. Graphical representation of quantitative RT-PCR analysis of SATB1 (B-i) and SATB2 (B-ii) relative gene expression in HCT116, HCT15 and HT29 colorectal cancer cell lines (2D) vs spheroids (3D), expression of actin was used as an internal control. (*, P<0.05; **,P<0.001; ***, P<0.0001).

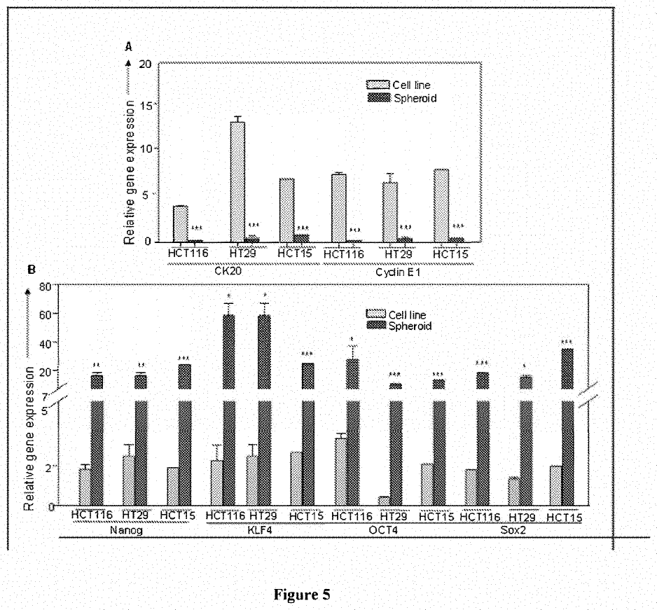

FIG. 5: Spheroids denote self-renewing fraction with elevated expression of pluripotency markers and downregulation of differentiation markers. Graphical representation of quantitative RT-PCR analysis of A-Differentiation markers (CK20 and cyclin E1); B-Pluripotency markers (Nanog, Klf4, Oct4 and Sox2) in HCT116, HCT15 and HT29 colorectal cancer cell lines (2D) vs spheroids (3D), expression of actin was used as an internal control. (*, P<0.05; **,P<0.001; ***, P<0.0001).

### DETAILED DESCRIPTION OF THE INVENTION

The invention will now be described in detail in connection with certain preferred and optional embodiments, so that various aspects thereof may be more fully understood and appreciated.

Aberrant expression of SATB family chromatin organizers and its association with an array of cancer types suggest its involvement in modulation of higher-order chromatin organization leading to carcinogenesis. SATB proteins provide structural network to regulate an array of genes hence its aberrant expression might lead to accumulation of molecular events culminating in tumorigenesis.

In a preferred embodiment, the present invention provides an in-vitro method for evaluating the progression and aggressiveness of cancer in a subject characterized by evaluating the combined expression patterns of SATB (Special AT-rich Sequence-Binding Protein) chromatin organizers, i.e., SATB-1 and SATB-2, which comprises;
(i) culturing tumorigenic cell cultures isolated from a primary tumor tissue, and
(ii) evaluating the combined expression levels of SATB-1 and SATB-2 chromatin organizers in the said cultures by RNA isolation and sequencing, quantitative RT-PCR, immunohistochemistry and immunoblotting.

The techniques of RNA isolation and sequencing, quantitative RT-PCR, immunohistochemistry and immunoblotting are well-established in the art and can be accordingly performed.

The present method provides for evaluating the progression and aggressiveness of cancer in cancer cell types selected from the group comprising urothelial bladder carcinoma, cervical squamous cell carcinoma, glioblastoma, kidney renal clear cell carcinoma, kidney renal papillary cell carcinoma, kidney renal papillary cell carcinoma, acute myeloid leukemia, low-grade glioma, liver hepatocellular carcinoma, lung adenocarcinoma, lung squamous cell carcinoma, ovarian cancer, pancreatic adenocarcinoma, sarcoma, skin cutaneous melanoma, stomach adenocarcinoma, uterine corpus endometrial carcinoma, colon adenocarcinoma, rectal adenocarcinoma and breast invasive carcinoma.

In an embodiment, the present invention provides a process for establishing the dual expression pattern of SATB family chromatin organizers towards cancer prognosis in an array of cancers from patient survival data, the said process comprising;
(a) performing Kaplan Meier survival analysis of TCGA (The Cancer Genome Atlas) dataset of patients from a plurality of cancer types comparing patient survival data to gene expression of primary tumors obtained through RNA sequencing analysis of TCGA dataset.
(b) correlating gene expression of SATB-1 and SATB-2 chromatin organizers obtained through RNA sequencing in step (a) to evaluate its significance towards cancer prognosis.

The first step involves performing Kaplan Meier survival analysis of correlation of expression of t SATB1 and SATB2 family chromatin organizers with patient survival for an array of tumor types. To evaluate association of SATB chromatin organizer expression within a tumour with the patient survival in an array of tumor types, TCGA data was used. Survival analysis was performed of TCGA dataset of patients from 14 cancer types in which gene expression obtained through RNA sequencing was correlated with patient survival data to evaluate significance of the two members of the SATB family chromatin organizers towards cancer prognosis. Patients were sorted based on gene expression (read counts of RSEM RNAseqV2 normalized) from highest to least. Top 15-25% and bottom 15-25% patients based on SATB1 expression were characterized as SATB1″ and SATB1^{low}, similarly patients were sorted based on gene expression profiles as SATB2^{hi} and SATB2^{low}. Statistical significance was calculated using logrank p-value≤0.05 considered as significant. However, those cancer types with lower patient numbers were considered for survival analysis (p value≤0.1) if they followed a survival/trend since increase in number of patients would lead to improve significance of the study. Survival studies clearly revealed that SATB chromatin organizers exhibit dynamic expression pattern, tissue specific regulation and are determinant of patient survival.

The cancer cell types analyzed to establish the dual expression pattern of SATB family chromatin organizers are selected from the group comprising urothelial bladder carcinoma, cervical squamous cell carcinoma, glioblastoma, kidney renal clear cell carcinoma, kidney renal papillary cell carcinoma, kidney renal papillary cell carcinoma, acute myeloid leukemia, low-grade glioma, liver hepatocellular carcinoma, lung adenocarcinoma, lung squamous cell carcinoma, ovarian cancer, pancreatic adenocarcinoma, sarcoma, skin cutaneous melanoma, stomach adenocarcinoma, uterine corpus endometrial carcinoma, colon adenocarcinoma, rectal adenocarcinoma and breast invasive carcinoma.

In one preferred embodiment, the present invention provides correlations between the status of expression of both SATB1 and SATB2 and the cancer type which serves as a highly reliable prognostic marker as established by the Kaplan Meier analysis based on patient survival data.

The present invention provides comparing the expression patterns with a chart indicating the expression levels of SATB-1 and SATB-2 chromatin organizers in a plurality of cancer cell types, the said chart comprising;
i. Increased combined expression of SATB1 and SATB2 correlates with poor patient survival for cervical squamous cell carcinoma, sarcoma and stomach adenocarcinoma;
ii. Increased SATB1 expression with decreased SATB2 culminates in poor patient survival for urothelial bladder carcinoma colon adenocarcinoma (COAD), rectal adenocarcinoma (READ), lung squamous cell carcinoma, uterine corpus endometrial carcinoma and liver hepatocellular carcinoma; and
iii. Increased expression of SATB2 and decreased expression of SATB1 in case of acute myeloid leukaemia, low-grade glioma, skin cutaneous melanoma and pancreatic adenocarcinoma, were determinant of poor patient survival. (FIG. 1; FIG. 2).

In another embodiment, the present invention provides an increased combined expression of SATB-1 and SATB-2 in a tumor sample compared to a healthy tissue which correlates with poor patient survival for cervical squamous cell carcinoma, sarcoma and stomach adenocarcinoma.

In yet another embodiment, the present invention provides an increased SATB1 expression with decreased SATB2 in a tumor sample compared to a healthy tissue which correlates with poor patient survival for urothelial bladder carcinoma colon adenocarcinoma (COAD), rectal adenocarcinoma (READ), lung squamous cell carcinoma, uterine corpus endometrial carcinoma and liver hepatocellular carcinoma

In one more embodiment, the present invention provides an increased expression of SATB2 and decreased expression of SATB1 in a tumor sample compared to a healthy tissue which correlates with poor patient survival for acute myeloid leukaemia, low-grade glioma, skin cutaneous melanoma and pancreatic adenocarcinoma.

Accordingly, the said chart indicating the combined expression levels of SATB1 and SATB2 in plurality of cancer cell types is defined in FIG. 3. The present method for evaluating cancer progression by determining the levels of combined expression of SATB-1 and SATB-2 in specific cancer cell types is an accurate and precise method.

In another preferred embodiment, the present invention provides functional significance associated with expression of SATB family chromatin organizers in terms of tumor regenerative potential.

The present invention provides the use of an in-vitro method for evaluating the progression and aggressiveness in cancer types selected from the group comprising urothelial bladder carcinoma, cervical squamous cell carcinoma, glioblastoma, kidney renal clear cell carcinoma, kidney renal papillary cell carcinoma, kidney renal papillary cell carcinoma, acute myeloid leukemia, low-grade glioma, liver hepatocellular carcinoma, lung adenocarcinoma, lung squamous cell carcinoma, ovarian cancer, pancreatic adenocarcinoma, sarcoma, skin cutaneous melanoma, stomach adenocarcinoma, uterine corpus endometrial carcinoma, colon adenocarcinoma, rectal adenocarcinoma and breast invasive carcinoma, wherein, on determining the combined expression pattern of SATB-1 and SATB-2 in tumorigenic cells isolated from a primary tumor tissue of a subject/patient diagnosed with cancer, the risk of the subject or patient is inferred as follows;.
(i) Increased combined expression of SATB1 and SATB2 correlates with poor patient survival for cervical squamous cell carcinoma, sarcoma and stomach adenocarcinoma;
(ii) Increased SATB1 expression with decreased SATB2 culminates in poor patient survival for urothelial bladder carcinoma colon adenocarcinoma (COAD), rectal adenocarcinoma (READ), lung squamous cell carcinoma, uterine corpus endometrial carcinoma and liver hepatocellular carcinoma; and
(iii) Increased expression of SATB2 and decreased expression of SATB1 in case of acute myeloid leukaemia, low-grade glioma, skin cutaneous melanoma and pancreatic adenocarcinoma, were determinant of poor patient survival.

In one preferred embodiment, the present invention provides an in-vitro method for evaluating the progression and aggressiveness of colorectal cancer/colon adenocarcinoma/Rectal adenocarcinoma in a subject, characterized by evaluating the combined expression patterns of SATB (Special AT-rich Sequence-Binding Protein) chromatin organizers, i.e., SATB-1 and SATB-2, which comprises;
(i) culturing tumorigenic cell cultures isolated from a primary tumor tissue, and
(ii) evaluating the combined expression levels of SATB-1 and SATB-2 chromatin organizers in the said cultures by RNA isolation and sequencing, quantitative RT-PCR, immunohistochemistry and immunoblotting;

wherein the combined expression pattern of SATB indicates that an increase in SATB-1 expression with a decrease in SATB-2 expression in a tumorigenic cell cultured as a 3D spheroid culminates in poor patient survival in a subject diagnosed with colorectal cancer/colon adenocarcinoma/Rectal adenocarcinoma.

FIG. 4 indicates an increase in the SATB1 expression levels and a reduction in the SATB-2 expression levels in case of colorectal cell lines cultured as spheroid cultures. Therefore, the present in-vitro method is a foolproof method to evaluate patient survival by determining the expression of SATBs.

Accordingly, the present invention provides a process for profiling combined expression pattern of SATB (special AT-rich sequence-binding protein) family chromatin organizers i.e. SATB-1 and SATB-2 as a biomarker tool for cancer prognosis comprising with a proof of principle based on functional assay of in-vitro studies;
(a) constructing 3D and 2D cultures from cancer cell lines and
(b) Profiling gene expression pattern of SATB family chromatin organizers using quantitative RT-PCR in the said cancer cell line generated spheroids (3D culture) and cell lines (2D culture).

In this context, spheroids (3D cultures) from colorectal cancer cell lines were generated which are established functional models to study regenerative cells and the expression of SATB1 and SATB2 across the 3D cultures was evaluated. Spheroids essentially comprise functional isolation of tumor initiating cells (TICs) or regenerative cells which relies on diverse characteristics such as anchorage independent growth, chemoresistance, self-renewal, asymmetric division, and pluripotency. Spheroids mirror the 3D cellular context and relevant pathophysiological gradients of in vivo tumors. Hence, spheroid (3D) is an appropriate in vitro model system to evaluate cancer aggressiveness.

It was observed that SATB1 was up-regulated in spheroid cultures and down-regulated in cell lines on the other hand SATB2 was down-regulated in 3D vs 2D cultures. This suggests that there exists a balance between expression of SATB chromatin organizers which governs intratumoral cellular and molecular functions.

In another preferred embodiment, the present invention provides a kit for evaluating the progression of cancer in a subject by estimating the dual expression pattern of SATB family chromatin organizers towards cancer prognosis comprising a chart indicating the expression levels of SATB-1 and SATB-2 chromatin organizers in a plurality of cancer cell types.

The prognostic kit of the present invention for evaluating the progression and aggressiveness of a cancer type to predict the survival in a subject diagnosed with cancer comprises;
(i) a healthy cell culture as a negative control
(ii) a tumorigenic cell culture specific to the cancer type as a positive control;
(iii) tools for performing RNA isolation, sequencing and RT-PCR; and
(iv) a chart stating the combined expression patterns of SATB1 and SATB2 specific to a cancer type indicating;
   (a) a combined increased expression of SATB1 and SATB2 correlates with poor patient survival for cervical squamous cell carcinoma, sarcoma and stomach adenocarcinoma;
   (b) an increased SATB1 expression with decreased SATB2 correlates with poor patient survival for urothelial bladder carcinoma, colon adenocarcinoma (COAD), rectal adenocarcinoma (READ), lung squamous cell carcinoma, uterine corpus endometrial carcinoma and liver hepatocellular carcinoma; and
   (c) an increased expression of SATB2 and decreased expression of SATB1 correlates with poor patient survival for acute myeloid leukaemia, low-grade glioma, pancreatic adenocarcinoma and Skin Cutaneous Melanoma.

In keeping with the aforesaid embodiment and the present in-vitro process, the present invention provides a healthy cell culture (negative control) and a tumorigenic cell line (positive control) that is specific to the cancer type which is to be evaluated in a patient for its aggressiveness and progression. Alternatively, the present in-vitro process can be done by directly using patient derived tissues, i.e. biopsy samples. The stages of RNA isolation and sequencing are performed according to the methods known in the art and can be performed by one skilled in the art. On determining the expression of SATB-1 and SATB-2 in a test sample, the expression pattern vis-à-vis the chart depicting the said patterns is analysed for patient survival.

Therefore, the present process indicating the combined expression pattern of SATB1 and SATB2 provides improved insights towards understanding tumour progression and therefore establishes it as a reliable prognostic marker.

The following examples are given by way of illustration of the present invention and therefore should not be construed to limit the scope of the present invention.

### Example 1: SATB Chromatin Organizers as Biomarkers for Cancer Prognosis

Survival analysis revealed that higher expression of both SATB1 and SATB2 was strongly correlated with poor patient survival for cervical squamous cell carcinoma, sarcoma and stomach adenocarcinoma. Correlation of SATB1 is linked with aggressiveness in case of stomach adenocarcinoma and cervical squamous cell carcinoma by Wang et al Jpn J Clin Oncol. 2015 and Hedner et al Virchows Arch. 2014, however none of these studies had reported association of SATB2. These findings are in agreement with respect to earlier reports for sarcoma by Wang et al Tumour Biol. 2015; Zhang et al Mol Cell Biochem. 2013 and Liu et al Mol Cell Biochem. 2017 wherein expression of SATB1 and SATB2 was individually correlated to tumor aggressiveness in independent studies.

SATB1 expression correlated negatively with SATB2 which culminates in poor patient survival for colon adenocarcinoma (COAD), rectal adenocarcinoma (READ), lung squamous cell carcinoma, uterine corpus endometrial carcinoma and liver hepatocellular carcinoma (Summarized in Table 1), which is speculated in case of earlier two cancer types by Brocato et al. (Carcinogenesis, 2015) and Tsuji et al. (Cancer Res. 2009), whereas the correlation with latter types is novel. Survival analysis revealed that in case of acute myeloid leukemia, low-grade glioma, urothelial bladder carcinoma and pancreatic adenocarcinoma higher expression of SATB2 and lower expression of SATB1 were determinant of poor patient survival (FIG. 1; FIG. 2). However, a recent study exhibited lack of clarity for correlation of expression of SATB1 for urothelial bladder carcinoma (Choudhary et al. Urol Oncol. 2018). Surprisingly, higher expression of both the SATB proteins conferred better patient survival for kidney renal clear cell carcinoma. The present analysis is in agreement with the established fact of downregulation of both SATB chromatin organizers in kidney renal clear cell carcinoma by Kowalczyk et al. (Cancer Genomics Proteomics, 2016); Guo et al. (Int J Clin Exp Pathol. 2015). Thus, the analysis presented here clearly indicates tissue specific regulation of SATB chromatin organizers with respect to tumor aggressiveness. This comprehensive analysis across multiple tumor types strongly argues in favour of exploiting both members of the SATB family chromatin organizers to categorize cancer types, which can be effectively used to design molecularly targeted therapy.

### Example 2: SATB Family Chromatin Organizers Exhibit Dynamic Regulation in Colorectal Cancer

Source of Biological Material:

HCT116, HCT15 cell lines were procured from European Collection of Cell Cultures (ECACC, SIGMA, St Loius, USA) and HT29 cell line was procured from American Type Culture Collection (ATCC, Manssas, Va., USA). SATB (Special AT-rich binding protein) family proteins have emerged as key regulators that integrate higher-order chromatin organization with regulation of gene expression. Recent studies have demonstrated that both SATB1 and SATB2 are instrumental for cancer progression. However, none of studies till date have performed extensive characterization of both members of SATB family chromatin organizers in terms of their role in colorectal cancer aggressiveness.

To evaluate regulation of SATB chromatin organizers in terms of regenerative potential 3D spheroid cultures from colorectal cancer cell lines (FIG. 4A-i, B-i, C-i) were established. Spheroids were generated 7 days after seeding of single colorectal cancer cells in ultra low attachment plate. The spheroids and corresponding cell lines, performed RNA isolation and profiled expression of SATB chromatin organizers using quantitative RT-PCR. A drastic increase in expression of SATB1 whereas a stark decrease in SATB2 in spheroid cultures as compared to their respective cell lines was observed (FIG. 4B-i, B-ii). Thus, expression of SATB1 positively correlated with spheroid phenotype which is an in vitro model for assessment of aggressiveness of cancer cells. In contrast, expression of SATB2 correlated negatively with spheroids.

Further, to understand the function the expression of pluripotency markers (Nanog, Klf4, Oct4, Sox2) and differentiation markers (CK20 and cyclinE1) across spheroid and corresponding 2D cell line cultures was evaluated. Spheroids represent the cellular fraction involved in self-renewal hence exhibited upregulation of all the pluripotency markers and correspondingly downregulation of differentiation markers as compared to respective cell lines (FIGS. 4 A, B). This data suggests dynamic regulation of SATB chromatin organizers towards cellular plasticity, wherein SATB1 is involved in generation and maintenance of self-renewal state. Expression of SATB2 correlated with CK20 and cyclinE1, suggests that SATB2 would be involved in mediating differentiation of colorectal cancer cells.

This example clearly denotes distinct functions governed by the two members of the SATB family of chromatin organizers involved in colorectal cancer progression.

Advantages of Invention:
The present invention provides a dynamic balance between expression patterns of SATB family chromatin organizers, which is tumor type specific and serves as determinant of survival of the cancer patient.
The present cancer tissue specific regulation of SATB family chromatin organizers serves as a precise biomarker for cancer prognosis.
Improved assessment of cancer aggressiveness.

We claim:

1. An in-vitro method for evaluating the progression and aggressiveness of cancer in a subject, characterized by evaluating the combined expression levels of SATB-1 and SATB-2 (Special AT-rich Sequence-Binding Protein) chromatin organizers which comprises;
i. culturing tumorigenic cell cultures isolated from a primary tumor tissue, and
ii. evaluating the combined expression pattern of SATB-1 and SATB-2 chromatin organizers in the said cultures by RNA isolation and sequencing and quantitative RT-PCR.

2. The in-vitro method as claimed in claim 1, wherein tumorigenic cell are cultured as 3D spheroid cultures.

3. The in-vitro method as claimed in claim 1, wherein the combined expression levels of STAB-1 and SATB-2 is evaluated for cancers selected from the group comprising cervical squamous cell carcinoma, sarcoma, stomach adenocarcinoma, urothelial bladder carcinoma, colon adenocarcinoma (COAD), rectal adenocarcinoma (READ), lung squamous cell carcinoma, uterine corpus endometrial carcinoma and liver hepatocellular carcinoma, acute myeloid leukaemia, low-grade glioma, pancreatic adenocarcinoma and skin Cutaneous Melanoma

4. The in-vitro method as claimed in claim 3, wherein evaluating the combined expression pattern of STAB-1 and SATB-2 is selected from the group comprising;
(i) combined increased expression of SATB1 and SATB2 correlates with poor patient survival for cervical squamous cell carcinoma, sarcoma and stomach adenocarcinoma;
(ii) increased SATB1 expression with decreased SATB2 correlates with poor patient survival for urothelial bladder carcinoma, colon adenocarcinoma (COAD), rectal adenocarcinoma (READ), lung squamous cell carcinoma, uterine corpus endometrial carcinoma and liver hepatocellular carcinoma; and
(iii) increased expression of SATB2 and decreased expression of SATB1 correlates with poor patient survival for acute myeloid leukaemia, low-grade glioma, pancreatic adenocarcinoma and Skin Cutaneous Melanoma.

5. The in-vitro method as claimed in claim 1, wherein the combined expression pattern of STAB-1 and SATB-2 family chromatin organizers towards cancer prognosis is established by a process comprising;
(a) performing Kaplan Meier survival analysis of TCGA (The Cancer Genome Atlas) dataset of patients from a plurality of cancer types, and
(b) comparing patient survival data to gene expression obtained through RNA sequencing of tumor samples of plurality of cancer types from TCGA to evaluate the significance of SATB1 and SATB2 chromatin organizers towards cancer prognosis.

6. An in-vitro method for evaluating the progression and aggressiveness of colorectal cancer/colon adenocarcinoma/Rectal adenocarcinoma as claimed in claim 1 in a subject characterized by evaluating the combined expression patterns of SATB (Special AT-rich Sequence-Binding Protein) chromatin organizers, i.e., SATB-1 and SATB-2, which comprises;
(i) culturing tumorigenic cell cultures isolated from a primary tumor tissue, and
(ii) evaluating the combined expression levels of SATB-1 and SATB-2 chromatin organizers in the said cultures by RNA isolation and sequencing and quantitative RT-PCR;
wherein the combined expression pattern of SATB indicates that an increase in SATB-1 expression with a decrease in SATB-2 expression in a tumorigenic cell cultured as a 3D spheroid culminates in poor patient survival in a subject diagnosed with colorectal cancer/colon adenocarcinoma/Rectal adenocarcinoma.

7. A prognostic kit for evaluating the progression and aggressiveness of a cancer type in a subject diagnosed with cancer comprising;
(i) a healthy cell culture as a negative control
(ii) a tumorigenic cell culture specific to the cancer type as a positive control;
(iii) tools for performing RNA isolation, sequencing and RT-PCR; and
(iv) a chart stating the combined expression patterns of SATB1 and SATB2 specific to a cancer type indicating;
(a) a combined increased expression of SATB1 and SATB2 correlates with poor patient survival for cervical squamous cell carcinoma, sarcoma and stomach adenocarcinoma;
(b) an increased SATB1 expression with decreased SATB2 correlates with poor patient survival for urothelial bladder carcinoma, colon adenocarcinoma (COAD), rectal adenocarcinoma (READ), lung squamous cell carcinoma, uterine corpus endometrial carcinoma and liver hepatocellular carcinoma; and
(c) an increased expression of SATB2 and decreased expression of SATB1 correlates with poor patient survival for acute myeloid leukaemia, low-grade glioma, pancreatic adenocarcinoma and Skin Cutaneous Melanoma.
